(19) United States
(12) Patent Application Publication
(10) Pub. No.: US 2021/0008414 A1
(43) Pub. Date: Jan. 14, 2021

(54) A METHOD AND SYSTEM FOR MEASURING A ROWING PROFILE

(71) Applicant: BAE SYSTEMS plc, London (GB)

(72) Inventors: Daniel Bishop, Rochester Kent (GB); Timothy Dewitt, Rochester Kent (GB); Stuart John Kitching, Rochester Kent (GB); Mark Allison, Rochester Kent (GB)

(73) Assignee: BAE SYSTEMS plc, London (GB)

(21) Appl. No.: 16/977,972

(22) PCT Filed: Mar. 5, 2019
(86) PCT No.: PCT/GB2019/050601

§ 371 Date: Sep. 3, 2020

(30) Foreign Application Priority Data
Mar. 7, 2018 (GB) 1803645.9

(51) Int. Cl. A63B 24/00; A63B 69/06; B63H 16/04

(52) U.S. Cl. CPC A63B 24/0062; A63B 24/0003; A63B 69/06; B63H 16/04; A63B 2220/833; A63B 2220/40; A63B 2225/50; A63B 2220/51; A63B 2220/13; A63B 2220/803; A63B 2225/20; A63B 2220/72; A63B 2220/89

(57) ABSTRACT

A system for measuring a rower profile of a rower; the system comprising one or more sensors located on an oar for measuring one or more forces imparted on the oar by the rower in use at a predetermined time; one or motion tracking devices for determining a position of the oar in space at the predetermined time; a processing module for interfacing with the one or more sensors and a remote monitoring device; and determining the rower profile from the sensor data and the one or more motion tracking devices.

The present invention relates to a method and system for measuring a rowing profile of a rower, particularly but not exclusively using a novel type of system and method.

Rowing is a very competitive sport and the performance of the rower is continually being assessed in order to determine ways in which to improve the performance of the rower. In order to do this, measurements of the rowers' performance are analysed in real time to identify appropriate improvements.

It is known to use so called smart oars, which include an integrated system in which the performance of the rower can be assessed. These types of system have not proved to be particularly effective. Measurements and the movements of the structures within the rowing boat are generally performed by bond strain gauges and sensors. These are typically located on the outside of the structure and measure the movement between two points. This tends to be difficult to achieve, as it is difficult to adhere a sensor on the outside of a long pole such as an oar. The sensor can be knocked off or easily damaged. In addition, bonding to composite materials, such as carbon fibre and the like, of these types of sensors can be particularly difficult due to the nature of the materials.

In order to assess the performance of a rower it is particularly useful to measure the power output of the rower. There is also a need for monitoring the precise nature of a rower's stroke, to determine not only the power being generated but also to look at how the technique of the rower could be altered to provide a better power and overall performance. Thus, a need exists for an intelligent oar which can monitor the motion of the oar in a three dimensional space to enable measurement of parameters such as stroke rate, stroke length, feather angles, blade slip, blade depth etc. to be assessed.

Accordingly, one object of the present invention is to provide a measuring device or sensor to help in the identification of the performance of a rower and to determine manners in which that performance can be improved.

### SUMMARY

According to an aspect of the present invention there is provided a system for measuring a rower profile of a rower; the system comprising one or more sensors located on an oar for measuring one or more forces imparted on the oar by the rower in use at a predetermined time; one or motion tracking devices for determining a position of the oar in space at the predetermined time; a processing module for interfacing with the one or more sensors and a remote monitoring device; and determining the rower profile from the sensor data and the one or more motion tracking devices.

Preferably, the motion tracking device includes one or more of a gyroscope, an accelerometer and a magnetometer for determining attitude and motion of the oar in 3 Dimensions.

Preferably, the attitude and motion of the oar in 3 Dimensions of the oar is provided by a MEMS chip supplying 6 or 9 axes of measurement

Preferably, at least one of the sensors comprises a sensor in the handle for determining a hand position and a force imparted by the hand on the handle at the predetermined time.

Preferably, the handle of the oar includes a one or more sensors attached to the shaft of the oar and a covering over the sensors.

Preferably, the covering forms a keyed connection with the handle to prevent the covering moving relative to the handle and to absorb forces associated with squeezing the handle.

Preferably, at least one of the sensors comprises one or more sensors for measuring the flex of the oar.

Preferably, the sensor comprises a Hall Effect sensor.

Preferably, the sensor is adapted to measure flex in one of more axes.

According to an further aspect of the present invention there is provided an oar comprising: one or more sensors for measuring one or more forces imparted on the oar by a rower in use at a predetermined time; one or motion tracking devices for determining a position of the oar in space at the predetermined time; a communication module for communicating the results from the one or more sensors and the one or more tracking devices with a remote monitoring device, to thereby determine a rower profile based on the data from the one or more sensors and the one or more monitors.

Preferably, the motion tracking device includes one or more of a gyroscope, an accelerometer and a magnetometer for determining attitude and motion of the oar in 3 Dimensions.

Preferably, the attitude and motion of the oar in 3 Dimensions of the oar is provided by a MEMS chip supplying 6 or 9 axes of measurement

Preferably, at least one of the sensors comprises a sensor in a handle for determining a hand position and a force imparted by the hand on the handle at the predetermined time.

Preferably, the handle of the oar includes a one or more sensors attached to the shaft of the oar and a covering over the sensors.

Preferably, the covering forms a keyed connection with the handle to prevent the covering moving relative to the handle and to absorb forces associated with squeezing the handle.

According to an aspect of the present invention there is provided handle for use with the oar of another aspect of the present invention.

According to an aspect of the present invention there is provided a method of measuring a rower profile of a rower; the method comprising the steps of: measuring one or more forces imparted on the oar by the rower in use at a predetermined time; determining a position of the oar in space at the predetermined time; communicating the results from the one or more sensors and the one or more tracking devices with a central processing unit; wherein the central processing unit combines the data from the one or more sensors to and the one or more motion tracking devices for one or more predetermined times to determine the rower profile.

### DESCRIPTION OF THE DRAWINGS

FIG. 1 is a diagram showing a rowing oar, according to an embodiment of the present invention.

FIG. 2 is a diagram showing a handle and sleeve arrangement for a rowing oar, according to an embodiment of the present invention.

FIG. 3 is an internal view of the FIG. 2 arrangement, according to an embodiment of the present invention.

FIG. 4 is view showing sensors associated with a sweep rowing oar inner handle, according to an embodiment of the present invention.

FIG. 5 is a view showing an oar arc captured by a motion-tracking element, according to an embodiment of the present invention.

FIG. 6 is graph of oar force in Newtons against time, according to an embodiment of the present invention.

FIG. 7 is graph of hand force in Newtons against time, which is shown for a sweep oar for utilising both hands, according to an embodiment of the present invention.

FIG. 8 is a graph showing hand position of the inside hand of a sweep oar superimposed over the hand force. This graph depicts inner handle hand position during applied force, according to an embodiment of the present invention.

FIG. 9 is graph showing horizontal angle of the oar movement with blade force, according to an embodiment of the present invention.

### DESCRIPTION OF THE DRAWINGS

In general, the present invention relates to system and method which may be incorporated into a rowing oar to enable measurement and analysis of the movements and associated performance of a rower.

The present invention can stand alone or be combined with a bend monitor utilising a hall effect sensor as described in the patent application entitled “A METHOD AND SYSTEM FOR MEASURING THE BENDING OF ROWING OARS” or common date and applicant.

In broad terms, the present invention relates to a novel instrumented oar. This provides a monitoring facility allowing for rowing coaches to monitor the technique of rowing athletes. The solution can be implemented for both sweep and sculling oars.

The instrumented oar uniquely provides force measurements at both the oar blade and at the handles along with grip positions. This allows the system to compute the power output of the rower, as well as the rower's technique and distribution of forces between the hand positions. The oar monitors the motion of the oar in 3D space allowing parameters such as stroke rate, stroke length, feather angles, blade slip, blade depth, feather speed etc. to be assessed. The overall result of the measurements and analysis is to obtain a rowing profile of the rower which gives an indication of the power at different parts of the rowing stroke in order to identify the rowers' rowing power profile. From the measurements of the rowing power profile the high and low points of the power profile can be determined along with the manner in which the rower performs the stoke, including blade depth, catch angle, feather speed, stroke length etc. The combination of the stroke and power data can then be combined and analysed to enable the rower to change elements of the stroke to improve efficiency by subtle changes in the movement of hands and body to alter the stroke and thus the power output.

Captured data may be streamed wirelessly in real time to a local seat hub or other portable device either within the boat or any other suitable location. Data may also be logged to local memory within the oar for later analysis. A removable battery is used to eliminate down time between sessions due to recharging needs.

In broad terms, the data captured according to the present invention includes motion tracking data from one or more motion tracking sensors to determine for example the nature of each stoke of the oar. A stroke is the movement of the oar from the entry of the oar into the water in a first movement or stroke, to entry of the oar into the water for the start of a second stroke. This is a nominal definition of the term stroke, which will have a clear meaning to anyone skilled in the art. In addition, various sensors are used to measure force, temperature, movement or any other relevant parameter which enable the power profile of the stroke to be determined.

Each rower has a rowing profile, which is unique and varies from rower to rower. The rowing profile can be determined by the data measured by the oar sensors. Analysis of the rower profile can then provide detailed information about the nature of the stroke and action of the rower. In addition, a large number of technical and power related determinations can be made in respect of the stroke and that will allow a rower and their trainer to improve the rowing profile to best suit the rower, the boat and the team. Information that is germane to the nature of the rower profile, includes but is not limited to the following:

stroke signature, which is the general movement of the oar as the stroke occurs;

the point of entry into the water and exit from the water of the oar;

length of the stoke;

the catch and finish angles;

feathering and squaring speed;

the depth of the oar in the water;

the height above the water in the return movement;

oar skimming;

measurement of the differences in forces between the hands;

oar arc motion;

grip and hand motion;

angular movement of the blade;

blade entry and exit positions and angles;

amount of splash or clean entry profile;

catch slip angle; and

any other factor which induces drag and/or diminishes the power and optimal rower profile.

The force at an oar blade is determined by measuring the bend in the oar shaft. This may be achieved in a number of different methods. In a first example, a proprietary bend monitor based around a hall-effect sensor may be used as described in the co-pending patent application mentioned above. In this example, the sensor may be mounted either internally or externally to the oar's shaft.

In a second example, one or more strain gauges are attached to the oar shaft to detect horizontal and/or vertical bend.

The oar may have the above-mentioned sensors either alone or in combinations and may further include other sensors at any location which can communicate with the oar monitoring control system as will be described in greater detail below.

As the rower pulls the oar through the water data will be captured and recorded in real-time at a high sampling rate to enable the exact position of the oar to be determined at any moment in time. The flex and other movements of the oar shaft can be determined multiple times a second. In order to equate the flex to force and power measurements, the oar may be pre-calibrated to identify flex values captured by the sensors for specific weights loaded onto the oar blade.

The resultant oar force can then be plotted with respect to time and provide information to coaches and rowers alike to enable an analysis of performance and to determine how performance improvements can be achieved.

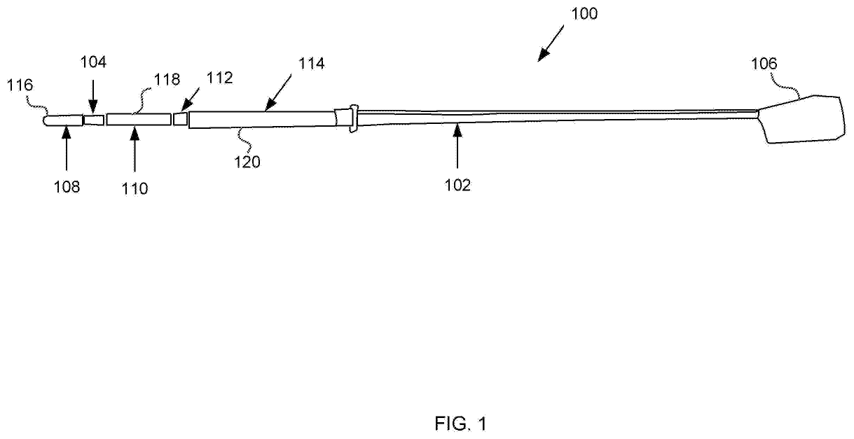

FIG. 1 shows a sweep rowing oar 100 having a shaft shown generally at 102, handles shown generally either side of 104 and a blade shown generally at 106. This is a sweep oar and has two separate areas 108 and 110, which are adapted to be held by each hand of the rower. In the case where the rower has two oars one in each hand, the arrangement according to the present invention can be adapted to accommodate this. For example, there may me sensors in both oars and each oar may be monitored to determine the forces on each oar. As will be appreciated either oar could be the same or one may have more complexity than the other. For example, a common monitoring and data storage module may be provided for each pair of oars to reduce the cost and complexity of installation of the monitoring module.

Returning to FIG. 1, the handle may include one or more strain gauges 112, 104 and 114. The strain gauges are used to calculate the hand position and the forces applied thereto. The end of the handle includes a customised covering 116 which contains further force sensors to calculate the hand position and forces. The outer customised covering ensures that any squeezing forces are absorbed by the covering and do not interfere with the measurement of forces imparted by the hand on the handle in the rowing movement of the rower. An outer sleeve 118 provides a similar function at the second hand position location. Another method of measuring hand position and force on the inner handle can be realised by mathematically analysing the data from all strain gauges, oar bend monitor and outer handle hand position. This option allows the removal of the inner handle outer sleeve and inner handle force sensors. The oar may include other forces sensors as previously suggested. For example, an internal Hall Effect sensor may be located within the oar shaft in regions such as 120 and 102. Magnetic or other force sensors and strain gauges may be located at any other location, which may yield force related data, such as for example in the vicinity of the gate.

There is no limit to the combination of sensors, numbers of sensors or the locations thereof. The sensors communicate with a central processing unit which can be located at any appropriate location. The central processing unit may include communications modules, sensor interfaces, memory module, motion tracking, battery monitoring and processing modules as required for the function they are to carry out. The central processing unit may be located within or attached to the oar. The processing function may also be offloaded to the boat, on the rower or in any other suitable place.

The instrumented oar allows the same outer handle and collar adjustments available on standard oars, this ensures that adjustments can be made to accommodate different boat types or rowers.

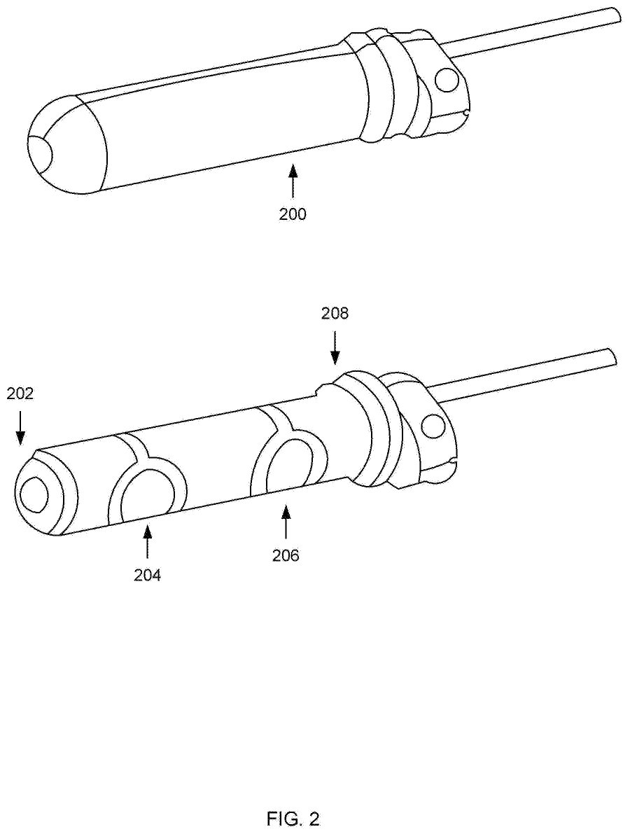

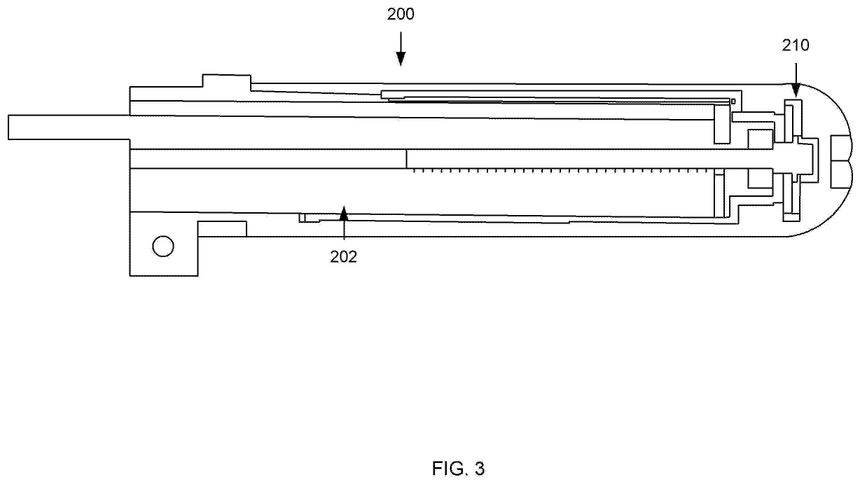

As is shown in FIGS. 2 and 3, the outside handle is designed to replace the existing standard outer handle. The original outer handle of the oar can be easily replaced with an instrumented version. This applies to both sweep and sculling oars. This allows the outside handle to be replaced with a custom handle containing force sensors. The outer customised covering 200 is adapted to fit snuggly over the end of the handle 202. The handle as previously indicated includes one or more force sensors 204, 206 attached thereto. The handle further includes a first part of a joint which includes external keying 208 which is adapted to cooperate with the internal keying 210 on the inside of the covering 200 as can be seen in FIG. 3 to ensure forces are directly applied to the force sensors without outer sleeve rotation.

In the example shown, the sensors comprise two force sensitive resistor (FSR) sensors underneath a sleeve. The sleeve eliminates any force due to squeezing of the grip and distributes force onto the sensors. The distribution of force between the FSR sensors can be used to determine the location of the centre of force that is being applied to sleeve and thus the rowers hand position.

The handle designs utilises keying to ensure force is applied directly onto the FSR sensors and to stop the outer sleeve from rotating. The end of the outside handle uses internal keying at one end to allow the rower to place their hand comfortably over the end of the handle.

Where the outside handle is not removable, a strain gauge can be placed on the oar shaft directly below the handle to measure flex and thus force applied to the handle. It is also possible to use a strain gauge in conjunction with the custom outside handle to provide increased accuracy on hand force. It is also possible to use a strain gauge below the outside handle to compute both hand force and hand position based upon the information provided by the strain gauge alone. This is computed by using the force data captured by the blade force measurements together with the readings taken from the strain gauge below the outside handle and strain gauge below the inside handle for a sweep oar.

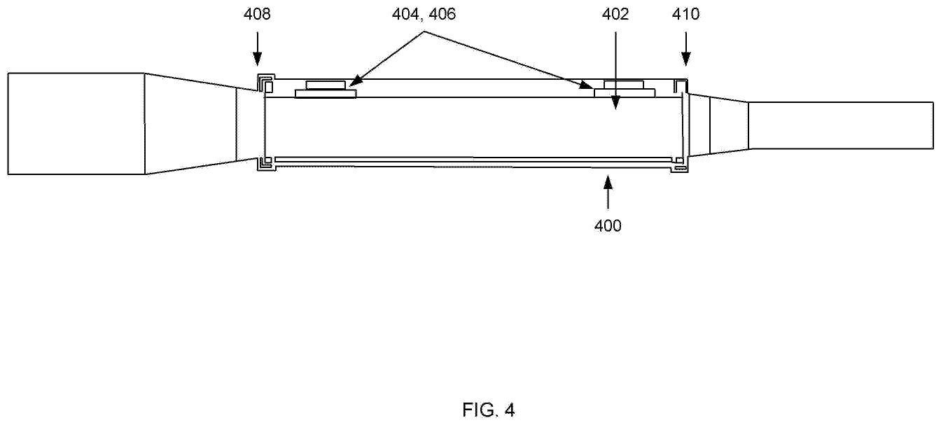

Grip force and hand position for inside handle on a sweep oar only is shown with respect to FIG. 4. The force and hand position measurements for the inside handle (sweep oar only) can be computed using a number of different methods. For a first method a custom sleeve 400 is attached over the inside handle 402 with two force sensors 404, 406 mounted to the shaft underneath the sleeve. The sleeve eliminates any force due to squeezing of the grip and distributes force onto the sensors. The distribution of force onto the FSR sensors can be used to determine the location of the centre of force that is being applied to sleeve and thus the rowers hand position.

FIG. 4 shows the inside handle 402 with the outer sleeve 400 pressing down on two force sensors 404, 406 located between the sleeve and the oar inner handle area. Two collars 408, 410 hold the sleeve in place on the tapered area of the oar shaft and contain keying to stop rotation of the outer sleeve. The inside handle may be pre-calibrated to identify force values captured by the sensors for specific forces applied to the handle.

A second method (not shown) utilizes a strain gauge located on the tapered part of the oar shaft below the inside handle and towards the blade. This strain gauge can be calibrated to measure the force on the inside handle alone by subtracting the imparted force applied by the outer handle previously measured.

The attitude and motion in 3D of the oar is provided by a MEMS chip supplying 6 or 9 axes of measurement derived from gyro, accelerometer and magnetometer sensor inputs which are captured multiple times a second. The MEMS chip may be located in any appropriate location in or on the oar. In one example, the MEMS chip may be located within a processing module that is strapped to the oar shaft. This information provides a continuous track of the oar position in space which allows parameters such as stroke rate, stroke length, feather angles, blade slip, blade depth, oar arc etc. to be determined. This data is then combined and possibly superimposed over the blade and hand forces to provide a complete picture of the rower's unique characteristics as will now be described.

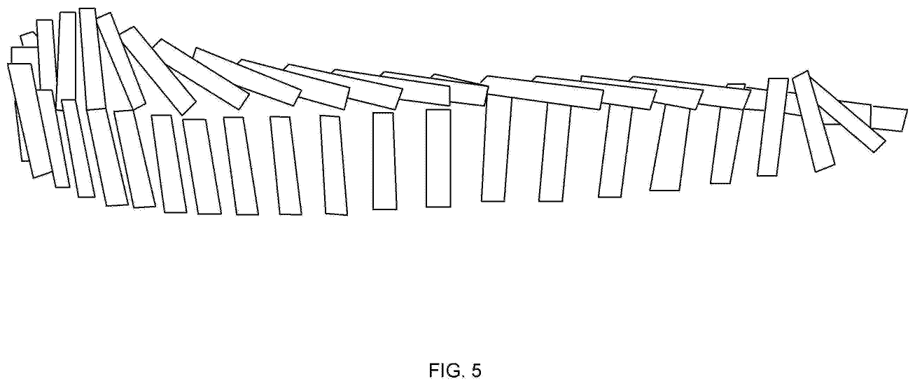

The image in FIG. 5 shows an oar arc captured by the motion tracking element of the design which may also be attached to the oar at an appropriate location.

The oars can include processing and memory devices to enable collection and/or processing of data received from each sensor over time. In addition, other sensors and measurements may be made and collected in conjunction with the oar sensor measurements, for example temperature to help mathematically null-out any effect of localised temperature swing in the proximity of the bend sensors.

The output of each sensor can be analysed to determine the flex and movement of the oar relative to it's “at rest position”. From known parameters of movements of the oar, for example, the amount of flex per kilogram it is possible to calculate the force imparted on the oar at any time. From these measurements and other data collected at the time or otherwise it is possible to build up an accurate and thorough assessment of the performance of a rower.

Measuring the force and hand position at the outside (for a sculling oar) or both handles (for a sweep oar) by the rower provides coaches with unique information regarding the rowers hand position and force distribution between his two hands. As the rower pulls the oar through the water strain gauges or custom hand sensors will record the force on the hand grip multiple times a second. The handle may be pre-calibrated to identify force or flex values captured by the sensors for specific forces applied to the handle.

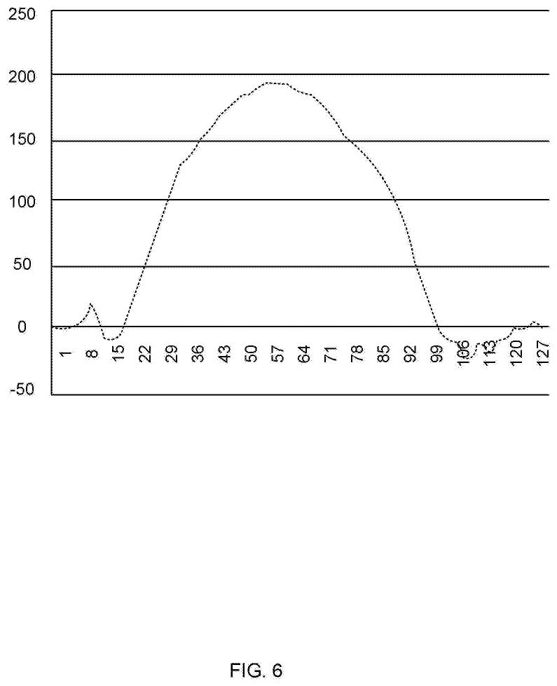

FIG. 6 shows a typical plot of oar force in Newtons against time.

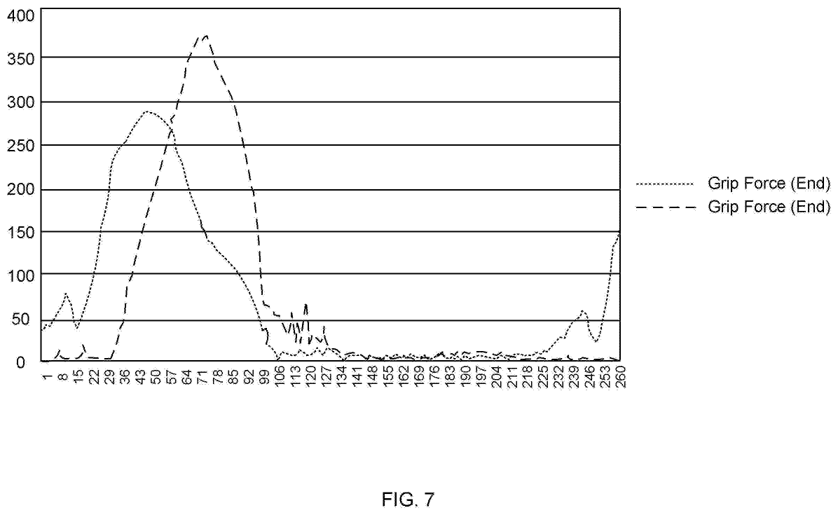

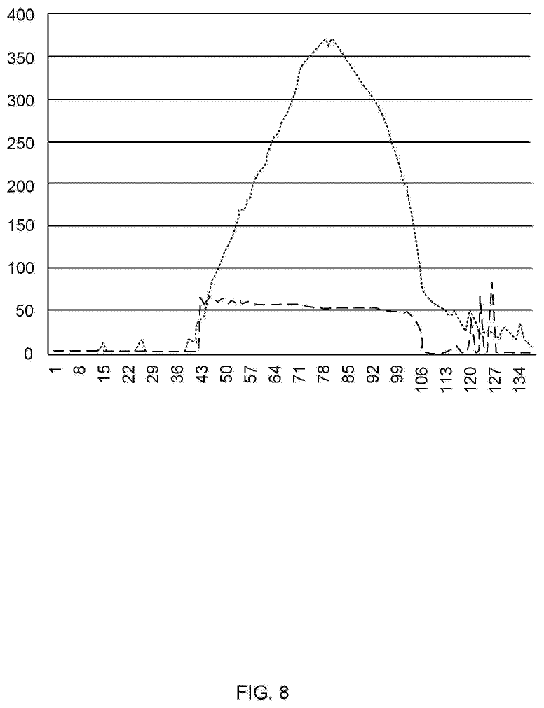

FIG. 7 shows how the resultant hand force can then be plotted with respect to time and provide a host of information to coaches. A typical plot of hand force in Newtons against time is shown for a sweep oar for both hands. The plot shows how force is distributed between the hands during the rowing stroke. Hand position is computed through either the measurement of force distribution on the embedded FSR sensors or computed from the strain gauge readings using the data from both handle strain gauges together with the force data from the blade which allows not only force to be calculated but also hand position. This is achievable by the characteristics of the strain gauge and the unique deformation of the carbon fiber shaft of the oar that occurs with changes of force and hand position. FIG. 8 shows hand position of the inside hand superimposed over the hand force showing a hand position mid grip (50%) during the applied force.

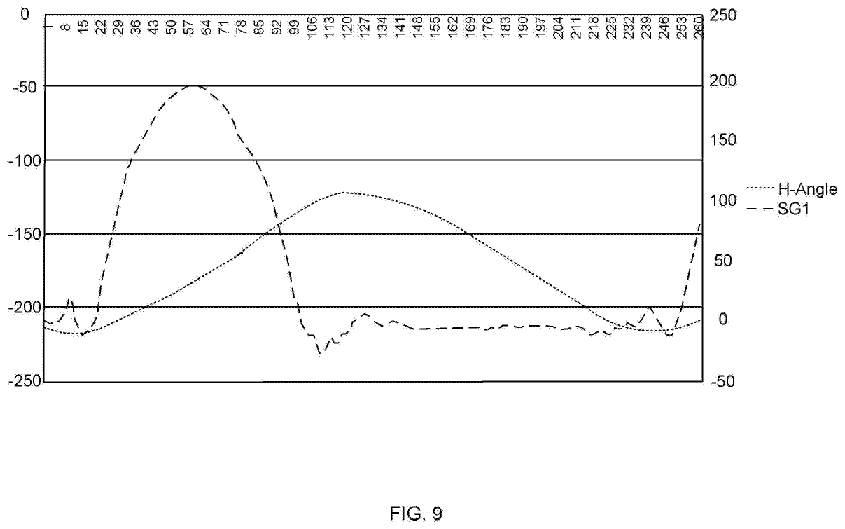

FIG. 9 shows how motion tracking can be used in conjunction with the force sensors to provide detailed information to the coaches on the rowers characteristics. For example, the graph shows the horizontal angle of the oar movement with the blade force. This allows the coaches to determine the point within the stroke where maximum force was applied, along with how quickly the force was applied when entering the water.

The power profile of each stoke can be analyzed and compared with an ideal or optimal profile or the profile of others. In so doing a determination can be made as to where the stroke has less than optimal power or when compared with another subject. Then by analysis of the movement that brought about the non-optimal power levels, the rower's movements can be adjusted through training or other means to move the rowing profile closer to the optimal profile. In the situation of multiple rowers in a boat the movements of all the rowers can be combined and the overall power profile determined for all users. This can enable the required changes for improvements to either individuals or the team as a whole to be determined. For example, if the boat is not moving in a straight-line changes to individuals and or their positions in the boat can be analyzed to determine the point at which the optimal profile of either the rower or the team and boat as a whole is achieved.

Comparison of graphs from the same and other rowers identifies unique signatures, which can support coaches and enable subtle technique changes to be imparted to the rowers, thereby improving power and performance metrics.

A small, battery powered module containing a microcontroller and interface circuitry to the system may be mounted either internal to the oar shaft (if access is available) or on the outside of the shaft. The battery may be in a removable module which also contains non-volatile memory for storing logged data and holding any configuration data that may be required (e.g. location of rowlock stop, location of end handle on shaft, rowlock height etc.). This also allows batteries to be changed between sessions to eliminate downtime for recharging and to enable any logged data of rowing trials to be downloaded.

A wireless (Bluetooth or other wireless protocol) transmitter may be fitted which will continuously stream live data from the system to a remote hub or other portable device to capture any data received by any of the sensors. Buttons to control power, initialisation and recording to local non-volatile memory may be included and there may be one or more LEDs to indicate battery, calibration and recording status.

As will be appreciated an oar is a long thin structure, such as a pole, which is exposed to stresses and movements as it is used. The present invention offers a way in which these movements can be measured and then used to determine the causes and effects of such movements. Accordingly, the oar sensors and measurement techniques and analysis can be used with other similar structures, for example a yacht mast or a pole vaulting pole, in the sports environment. Similarly, any long relatively thin structure that is subjected to stresses, strains and flexions may be sensed and analysed by the system and method of the present invention.

Although the present invention has been described in connection with some embodiments, it is not intended to be limited to the specific form set forth herein. Rather, the scope of the present invention is limited only by the accompanying claims. Additionally, although a feature may appear to be described in connection with particular embodiments, one skilled in the art would recognize that various features of the described embodiments may be combined in accordance with the invention. In the claims, the term ‘comprising’ does not exclude the presence of other elements or steps.

Furthermore, the order of features in the claims does not imply any specific order in which the features must be performed and in particular, the order of individual steps in a method claim does not imply that the steps must be performed in this order. Rather, the steps may be performed in any suitable order. In addition, singular references do not exclude a plurality. Thus, references to ‘a’, ‘an’, ‘first’, ‘second’, etc. do not preclude a plurality. In the claims, the term ‘comprising’ or “including” does not exclude the presence of other elements.

1. A system for measuring a rower profile of a rower, the system comprising:
one or more sensors located on an oar for measuring one or more forces imparted on the oar by the rower in use at a predetermined time;
one or more motion tracking devices for determining a position of the oar in space at the predetermined time; and
a processing module for interfacing with the one or more sensors and a remote monitoring device, and determining the rower profile based on data from the one or more sensors and the one or more motion tracking devices.

2. The system of claim 1, wherein the one or more motion tracking devices include one or more of a gyroscope, an accelerometer, and a magnetometer for determining attitude and motion of the oar in three dimensions.

3. The system of claim 2, wherein the attitude and motion of the oar in three dimensions is provided by a MEMS chip supplying 6 or 9 axes of measurement.

4. The system of claim 1, wherein the oar has a handle, and at least one of the one or more sensors comprises a sensor in the handle for determining a hand position and a force imparted by the hand on the handle at the predetermined time.

5. The system of claim 4, the system further including a covering over at least the at least one sensor in the handle.

6. The system of claim 5, wherein the covering forms a keyed connection with the handle to prevent the covering moving relative to the handle and to absorb forces associated with squeezing the handle.

7. The system of claim 1, wherein at least one of the one or more sensors comprises one or more sensors for measuring the flex of the oar.

8. The system of claim 7, wherein the at least one sensor comprises a Hall Effect sensor.

9. The system of claim 7, wherein the at least one sensor is adapted to measure flex in one of more axes.

10. An oar comprising:
one or more sensors for measuring one or more forces imparted on the oar by a rower in use at a predetermined time;
one or motion tracking devices for determining a position of the oar in space at the predetermined time; and
a communication module for communicating data from the one or more sensors and the one or more tracking devices to a monitoring device, to thereby determine a rower profile based on the data from the one or more sensors and the one or more tracking devices.

11. The oar of claim 10, wherein the one or more motion tracking devices include one or more of a gyroscope, an accelerometer, and a magnetometer for determining attitude and motion of the oar in three dimensions.

12. The oar of claim 11, wherein the attitude and motion of the oar in three dimensions of the oar is provided by a MEMS chip supplying 6 or 9 axes of measurement.

13. The oar of claim 10, wherein at least one of the one or more sensors comprises a sensor in a handle of the oar, the at least one sensor for determining a hand position and a force imparted by the hand on the handle at the predetermined time.

14. The oar of claim 10, wherein a handle of the oar includes at least one of the one or more sensors, the oar further comprising a covering over at least the at least one sensor.

15. The system of claim 14, wherein the covering forms a keyed connection with the handle to prevent the covering moving relative to the handle and to absorb forces associated with squeezing the handle.

16. The system of claim 13, wherein the handle is removably attachable, such that the handle can replace a standard handle of the oar.

17. A method of measuring a rower profile of a rower, the method comprising:
determining one or more forces imparted on the oar by the rower in use at a predetermined time, the one or more forces detected by one or more sensors on the oar;
determining a position of the oar in space at the predetermined time, the position detected by one or more motion tracking devices on the oar;
communicating data from the one or more sensors and the one or more motion tracking devices to a central processing unit;
wherein the central processing unit uses the data from the one or more sensors to and the one or more motion tracking devices for one or more predetermined times to determine the rower profile.

18. The method of claim 17, wherein the central processing unit is remote to the oar, and communicating the data includes communicating the data using a wireless communication protocol.

19. The system of claim 1, wherein the one or more sensors, the one or more motion tracking devices, and/or the processing module are included in a removable handle of the oar.

20. The system of claim 10, wherein the one or more sensors, the one or more motion tracking devices, and/or the communication module are included in a removable handle of the oar.
